(19) United States
(12) Patent Application Publication
(10) Pub. No.: US 2007/0213376 A1
(43) Pub. Date: Sep. 13, 2007

(54) Halogenated sulfonamide derivatives

(76) Inventors: Vrej Jubian, North Haledon, NJ (US); Mathivanan Packiarajan, Saddle Brook, NJ (US); Emily Reinhard, Ridgewood, NJ (US)

Correspondence Address: LUNDBECK RESEARCH USA, INC.;ATTENTION: STEPHEN G. KALINCHAK, LEGAL, 215 COLLEGE ROAD, PARAMUS, NJ, 07652, US

(21) Appl. No.: 11/714,008

(22) Filed: Mar. 5, 2007

Related U.S. Application Data
(60) Provisional application No. 60/779,765, filed on Mar. 7, 2006.
(60) Provisional application No. 60/878,327, filed on Jan. 3, 2007.

(51) Int. Cl. A61K 31/44

(52) U.S. Cl. 514/342; 546/270.4

(57) ABSTRACT

This invention is directed to halogenated sulfonamide derivatives which are ligands at the NPY Y5 receptor. The invention provides a pharmaceutical composition comprising a therapeutically effective amount of a compound of the invention and a pharmaceutically acceptable carrier. This invention also provides a pharmaceutical composition made by admixing a therapeutically effective amount of a compound of the invention and a pharmaceutically acceptable carrier. This invention further provides a process for making a pharmaceutical composition comprising combining a therapeutically effective amount of a compound of the invention and a pharmaceutically acceptable carrier. This invention also provides a method of treating a subject suffering from depression which comprises administering to the subject an amount of a compound of the subject invention. This invention also provides a method of treating a subject suffering from anxiety which comprises administering to the subject an amount of a compound of the subject invention. This invention also provides a method of treating a subject suffering from obesity which comprises administering to the subject an amount of a compound of the subject invention. Furthermore, the present invention is directed to use of a compound of the invention for the manufacture of a medicament for treating a subject suffering from depression, anxiety or obesity.

This application claims benefit of U.S. provisional application No. 60/779,765, filed Mar. 7, 2006 and U.S. provisional application No. 60/878,327, filed Jan. 3, 2007, the contents of both of which are hereby incorporated by reference into the subject application.

### FIELD OF THE INVENTION

The present invention relates to compounds that are ligands at the neuropeptide Y Y5 receptor, and as such are useful to treat disorders such as depression, anxiety and obesity.

### BACKGROUND OF THE INVENTION

Throughout this application, various publications are referenced to in full citations. The disclosures of these publications are hereby incorporated by reference into this application to describe more fully the state of the art to which this invention pertains.

Neuropeptide Y (NPY) is a 36 amino acid neuropeptide expressed in the peripheral and central nervous system. This peptide is a member of the pancreatic polypeptide family, which also includes pancreatic polypeptide (PP) and peptide YY (PYY). Moreover, the biological effects of NPY are mediated through its interaction with receptors that belong in the superfamily of G protein-coupled receptors.

Presently, five NPY receptor subtypes have been cloned: Y1 (D. Larhammar, et al., J. Biol. Chem., 1992, 267, 10935-10938); Y2 (C. Gerald, et al., J. Biol. Chem., 1995, 270, 26758-26761); Y4 (J. Bard, et al., J. Biol. Chem., 1995, 270, 26762-26765); Y5 (C. Gerald, et al., J. Biol. Chem., 1995, 270, 26758-26761); and y6 (P. Gregor, et al., J. Biol. Chem., 1996, 271, 27776-27781). All these receptor subtypes are expressed in several species except for the y6 subtype, which has been shown to be expressed in mouse and rabbit but not in rat and primate. A Y3 subtype has been proposed based on pharmacological data. However, the Y3 subtype has yet to be cloned and its existence remains to be fully established.

NPY exerts numerous physiological effects. On the basis of animal studies, it is evident that a contributory relationship exists between NPY and its receptors with disorders such as depression, anxiety and obesity. For instance, NPY expression is shown to be sensitive to energy status while NPY administration reduces energy expenditure, and another significant ability of NPY is to acutely stimulate feeding (S. Kalra, et al., Endocr. Rev., 1999, 20, 68-100). The NPY Y5 receptor has also been shown to be a receptor subtype responsible for NPY-induced food intake (C. Gerald, et al., Nature, 1996, 382, 168-171).

Additionally, the link between NPY and mood disorders such as depression and anxiety is established in the literature. For example, rats subjected to chronic mild stress exhibit anhedonia, a feature of clinical depression (P. Willner, et al., Eur. J. Pharmacol., 1997, 340, 121-132); they also contain elevated levels of NPY mRNA in hypothalamus accompanied by a reduction in hippocampus (V. Sergeyev, et al., Psychopharmacology, 2005, 178, 115-124). The behavioral changes associated with chronic mild stress are reversed by a variety of antidepressants (P. Willner, et al., Eur. J. Pharmacol., 1997, 340, 121-132). In one study of antidepressant therapies, rats treated with citalopram displayed an increased level of hippocampal NPY receptor binding with no change in NPY-like immunoreactivity (H. Husum, et al., Neuropsychopharmacology, 2001, 2, 183-191); conversely, electroconvulsive shock produced an increased level of hippocampal NPY-like immunoreactivity with no change in NPY receptor binding. These findings suggest that abnormal levels of NPY play a role in depressive illness, and that agents capable of regulating NPY and/or NPY receptor function particularly in limbic regions are useful for treating depression. Y5 is a NPY receptor expressed in limbic regions (M. Wolak, et al., J. Comp. Neurol., 2003, 22, 285-311; and K. Nichol, et al., J. Neurosci., 1999, 19, 10295-10304). Accordingly, agents capable of regulating Y5 receptor function are therefore predicted to be useful for treating depression.

Animal models of anxiety also reveal abnormal levels of NPY. In one example, maternally separated rats display an anxious and depressive phenotype throughout adulthood (R. Huot, Psychopharmacology, 2001, 158, 366-73); they also contain elevated levels of NPY-like immunoreactivity in hypothalamus accompanied by a reduction in hippocampus and cortex (P. Jimenez-Vasquez, Brain Res. Dev., 2001, 26, 149-152; H. Husum and A. Mathe, Neuropsychopharmacology, 2002 27:756-64; and H. Husum et al., Neurosci Lett., 2002, 333, 127-130). In a second example, rats subjected to fear conditioning display increased anxiety-like behavior; they also contain elevated levels of NPY in hypothalamus, amygdala and nucleus accumbens accompanied by a reduction in frontal cortex. The behavioral changes produced by fear conditioning can be reversed by treatment with anxiolytic drugs. In one study of fear conditioning, both the anxiety-like behavior and altered expression of NPY were reversed by treatment with diazepam (R. Krysiak, et al., Neuropeptides, 2000, 34, 148-57). These findings further suggest that NPY plays a role in anxiety, and that agents capable of regulating NPY and/or receptor function particularly in limbic regions are useful for treating anxiety. Y5 is a NPY receptor expressed in limbic regions (M. Wolak, et al., J. Comp. Neurol., 2003, 22, 285-311; and K. Nichol, et al., J. Neurosci., 1999, 19, 10295-10304). Accordingly, agents capable of regulating Y5 receptor function are therefore predicted to be useful for treating anxiety.

In our laboratories, the compounds of the invention have been evaluated in animal models predictive for antidepressant activity. It has been discovered that these compounds produce effects similar to that observed by known antidepressants.

Numerous groups have postulated various selective NPY Y5 small molecule ligands for the treatment of these disorders. In addition to possessing the appropriate pharmacological elements to move forward in the clinic, a compound should have favorable ADME properties such as metabolic stability. Issues relating to drug-drug interactions and toxicity should also be addressed. Metabolic stability of a drug may be predicted by its clearance rate in liver microsomes. Additionally, the cytochrome P450s (CYPs) play a role in metabolism, and CYP inhibition may be used to predict the potential risks of drug-drug interactions and/or toxicity.

Moreover, current treatments for depression, anxiety and obesity are on the market. However, numerous patients do not respond to current treatments. Hence, there remains the need for alternative methods of treatment.

### SUMMARY OF THE INVENTION

The objective of the present invention is to provide compounds that are ligands at the NPY Y5 receptor. The present invention relates to compounds of Formula I.

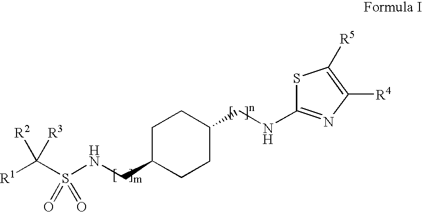

wherein R¹ is H or C₁-C₆ straight chained or branched alkyl;
wherein R² is C₁-C₆ straight chained or branched alkyl;
or wherein R¹, R² and the carbon to which they are attached may form C₃-C₆ cycloalkyl;
wherein R³ is H or methyl;
wherein R⁴ is 2-pyridyl or 3-pyridyl, wherein the 2-pyridyl or 3-pyridyl is substituted with one or more F, Cl, Br, I or CF₃, and optionally substituted with CH₃;
wherein R⁵ is H or methyl;
wherein m is an integer from 0 to 2 inclusive; and
wherein n is an integer from 0 to 2 inclusive;
or a pharmaceutically acceptable salt thereof.

In separate embodiments of the invention, the compound is selected from one of the specific compounds disclosed in the Experimental Section.

Furthermore, the present invention provides a pharmaceutical composition comprising a therapeutically effective amount of a compound of Formula I and a pharmaceutically acceptable carrier. The present invention also provides a process for making a pharmaceutical composition comprising admixing a therapeutically effective amount of a compound of Formula I and a pharmaceutically acceptable carrier.

Moreover, the present invention provides a method of treating a subject suffering from depression comprising administering to the subject a therapeutically effective amount of a compound of Formula I. The present invention further provides a method of treating a subject suffering from anxiety comprising administering to the subject a therapeutically effective amount of a compound of Formula I. The present invention further provides a method of treating a subject suffering from obesity comprising administering to the subject a therapeutically effective amount of a compound of Formula I.

Furthermore, the present invention is directed to the use of a compound as defined in Formula I for the manufacture of a medicament useful for treating depression. Additionally, the present invention is directed to the use of a compound as defined in Formula I for the manufacture of a medicament useful for treating anxiety. The present invention further provides for the use of a compound as defined in Formula I for the manufacture of a medicament useful for treating obesity.

### DETAILED DESCRIPTION OF THE INVENTION

### Definitions

In the present invention, the term “C₁-C₆ straight chained or branched alkyl” refers to a saturated hydrocarbon having from one to six carbon atoms inclusive. Examples of such substituents include, but are not limited to, methyl, ethyl, 1-propyl, 2-propyl, 1-butyl, 2-butyl, 2-methyl-2-propyl, 2-methyl-1-propyl, n-pentyl and n-hexyl. Similarly, the term “C₁-C₄ straight chained or branched alkyl” refers to a saturated hydrocarbon having from one to four carbon atoms inclusive. Examples of such substituents include, but are not limited to, methyl, ethyl and n-butyl.

Furthermore, the term “C₃-C₆ cycloalkyl” refers to a saturated cyclohydrocarbon ring having from three to six carbon atoms inclusive. Included within this term are cyclopropyl, cyclobutyl, cyclopentyl and cyclohexyl.

For illustrative purposes, and without limiting the invention, the compound of example 1b has the following structure:

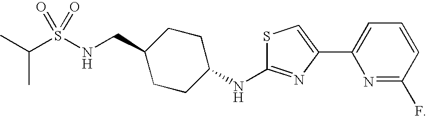

This compound is constructed from Formula I wherein R¹ and R² are methyl; R³ is H; m is 1; n is 0; R⁴ is 2-pyridyl substituted with F; and R⁵ is H.

Additionally, the invention further provides certain embodiments of the present invention that are described below.

In one embodiment, R³ is H; m is 0 or 1; and n is 0 or 1.

In another embodiment, R¹ is H or C₁-C₄ straight chained or branched alkyl.

In one embodiment, R² is C₁-C₄ straight chained or branched alkyl.

In a separate embodiment, R¹ is H, methyl or ethyl; and R² is methyl or ethyl.

In one embodiment, R¹ is methyl; R² is methyl; and R⁴ is 2-pyridyl or 3-pyridyl substituted with F or CF₃ and optionally substituted with CH₃.

In another embodiment, n is 1.

In one embodiment, m is 1.

In one embodiment, R⁴ is 3-pyridyl substituted with F; and R⁵ is H.

In one embodiment, R¹, R² and the carbon to which they are attached form C₃-C₆ cycloalkyl.

In one embodiment, R⁴ is 2-pyridyl or 3-pyridyl substituted with F.

In one embodiment, R¹, R² and the carbon to which they are attached form cyclopropyl or cyclobutyl; R⁴ is 3-pyridyl substituted with F; R⁵ is H; and n is 0.

In separate embodiments, the invention is directed to one of the specific compounds disclosed herein.

Furthermore, the present invention is directed to the use of a compound as defined above for the manufacture of a medicament useful for treating depression. Additionally, the present invention is directed to the use of a compound as defined above for the manufacture of a medicament useful for treating anxiety. The present invention further provides for the use of a compound as defined above for the manufacture of a medicament useful for treating obesity.

### Pharmaceutically Acceptable Salts

The present invention also comprises salts of the present compounds, typically, pharmaceutically acceptable salts. Such salts include pharmaceutically acceptable acid addition salts. Acid addition salts include salts of inorganic acids as well as organic acids.

Representative examples of suitable inorganic acids include hydrochloric, hydrobromic, hydroiodic, phosphoric, sulfuric, sulfamic, nitric acids and the like. Representative examples of suitable organic acids include formic, acetic, trichloroacetic, trifluoroacetic, propionic, benzoic, cinnamic, citric, fumaric, glycolic, itaconic, lactic, methanesulfonic, maleic, malic, malonic, mandelic, oxalic, picric, pyruvic, salicylic, succinic, methane sulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, theophylline acetic acids, as well as the 8-halotheophyllines (for example, 8-bromotheophylline and the like). Further examples of pharmaceutically acceptable inorganic or organic acid addition salts include the pharmaceutically acceptable salts listed in S. M. Berge, et al., J. Pharm. Sci., 1977, 66, 2, the contents of which are hereby incorporated by reference.

Furthermore, the compounds of this invention may exist in unsolvated as well as in solvated forms with pharmaceutically acceptable solvents such as water, ethanol and the like.

Racemic forms may be resolved into the optical antipodes by known methods, for example, by separation of diastereomeric salts thereof with an optically active acid, and liberating the optically active amine compound by treatment with a base. Separation of such diastereomeric salts can be achieved, e.g. by fractional crystallization. The optically active acids suitable for this purpose may include, but are not limited to d- or l-tartaric, madelic or camphorsulfonic acids. Another method for resolving racemates into the optical antipodes is based upon chromatography on an optically active matrix. The compounds of the present invention may also be resolved by the formation and chromatographic separation of diastereomeric derivatives from chiral derivatizing reagents, such as, chiral alkylating or acylating reagents, followed by cleavage of the chiral auxiliary. Any of the above methods may be applied either to resolve the optical antipodes of the compounds of the invention per se or to resolve the optical antipodes of synthetic intermediates, which can then be converted by methods described herein into the optically resolved final products which are the compounds of the invention.

Additional methods for the resolution of optical isomers, known to those skilled in the art, may be used. Such methods include those discussed by J. Jaques, A. Collet and S. Wilen in Enantiomers, Racemates, and Resolutions, John Wiley and Sons, New York, 1981. Optically active compounds can also be prepared from optically active starting materials.

### Pharmaceutical Compositions

The present invention further provides a pharmaceutical composition comprising a therapeutically effective amount of a compound of Formula I and a pharmaceutically acceptable carrier. The present invention also provides a pharmaceutical composition comprising a therapeutically effective amount of one of the specific compounds disclosed in the Experimental Section and a pharmaceutically acceptable carrier.

The compounds of the invention may be administered alone or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19^{th} Edition, Gennaro, Ed., Mack Publishing Co., Easton, Pa., 1995.

The pharmaceutical compositions may be specifically formulated for administration by any suitable route such as oral, rectal, nasal, pulmonary, topical (including buccal and sublingual), transdermal, intracisternal, intraperitoneal, vaginal and parenteral (including subcutaneous, intramuscular, intrathecal, intravenous and intradermal) routes. It will be appreciated that the route will depend on the general condition and age of the subject to be treated, the nature of the condition to be treated and the active ingredient.

Pharmaceutical compositions for oral administration include solid dosage forms such as capsules, tablets, dragees, pills, lozenges, powders and granules. Where appropriate, the compositions may be prepared with coatings such as enteric coatings or they may be formulated so as to provide controlled release of the active ingredient such as sustained or prolonged release according to methods well known in the art. Liquid dosage forms for oral administration include solutions, emulsions, suspensions, syrups and elixirs.

Pharmaceutical compositions for parenteral administration include sterile aqueous and nonaqueous injectable solutions, dispersions, suspensions or emulsions as well as sterile powders to be reconstituted in sterile injectable solutions or dispersions prior to use.

Other suitable administration forms include, but are not limited to, suppositories, sprays, ointments, creams, gels, inhalants, dermal patches and implants.

Typical oral dosages range from about 0.001 to about 100 mg/kg body weight per day. Typical oral dosages also range from about 0.01 to about 50 mg/kg body weight per day. Typical oral dosages further range from about 0.05 to about 10 mg/kg body weight per day. Oral dosages are usually administered in one or more dosages, typically, one to three dosages per day. The exact dosage will depend upon the frequency and mode of administration, the sex, age, weight and general condition of the subject treated, the nature and severity of the condition treated and any concomitant diseases to be treated and other factors evident to those skilled in the art.

The formulations may also be presented in a unit dosage form by methods known to those skilled in the art. For illustrative purposes, a typical unit dosage form for oral administration may contain from about 0.01 to about 1000 mg, from about 0.05 to about 500 mg, or from about 0.5 to about 200 mg.

For parenteral routes such as intravenous, intrathecal, intramuscular and similar administration, typical doses are in the order of half the dose employed for oral administration.

The present invention also provides a process for making a pharmaceutical composition comprising admixing a therapeutically effective amount of a compound of Formula I and a pharmaceutically acceptable carrier. In an embodiment of the present invention the compound utilized in the aforementioned process is one of the specific compounds disclosed in the Experimental Section.

The compounds of this invention are generally utilized as the free substance or as a pharmaceutically acceptable salt thereof. One example is an acid addition salt of a compound having the utility of a free base. When a compound of Formula I contains a free base such salts are prepared in a conventional manner by treating a solution or suspension of a free base of Formula I with a molar equivalent of a pharmaceutically acceptable acid. Representative examples of suitable organic and inorganic acids are described above.

For parenteral administration, solutions of the compounds of Formula I in sterile aqueous solution, aqueous propylene glycol, aqueous vitamin E or sesame or peanut oil may be employed. Such aqueous solutions should be suitably buffered if necessary and the liquid diluent first rendered isotonic with sufficient saline or glucose. The aqueous solutions are particularly suitable for intravenous, intramuscular, subcutaneous and intraperitoneal administration. The compounds of Formula I may be readily incorporated into known sterile aqueous media using standard techniques known to those skilled in the art.

Suitable pharmaceutical carriers include inert solid diluents or fillers, sterile aqueous solutions and various organic solvents. Examples of solid carriers include lactose, terra alba, sucrose, cyclodextrin, talc, gelatin, agar, pectin, acacia, magnesium stearate, stearic acid and lower alkyl ethers of cellulose. Examples of liquid carriers include, but are not limited to, syrup, peanut oil, olive oil, phospholipids, fatty acids, fatty acid amines, polyoxyethylene and water. Similarly, the carrier or diluent may include any sustained release material known in the art, such as glyceryl monostearate or glyceryl distearate, alone or mixed with a wax. The pharmaceutical compositions formed by combining the compounds of Formula I and a pharmaceutically acceptable carrier are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units such as capsules or tablets, each containing a predetermined amount of the active ingredient, and optionally a suitable excipient. Furthermore, the orally available formulations may be in the form of a powder or granules, a solution or suspension in an aqueous or non-aqueous liquid, or an oil-in-water or water-in-oil liquid emulsion.

If a solid carrier is used for oral administration, the preparation may be tabletted, placed in a hard gelatin capsule in powder or pellet form or it may be in the form of a troche or lozenge. The amount of solid carrier will vary widely but will range from about 25 mg to about 1 g per dosage unit.

If a liquid carrier is used, the preparation may be in the form of a syrup, emulsion, soft gelatin capsule or sterile injectable liquid such as an aqueous or non-aqueous liquid suspension or solution.

### Treatment of Disorders

As mentioned above, the compounds of Formula I are ligands at the NPY Y5 receptor. The present invention provides a method of treating a subject suffering from depression which comprises administering to the subject a therapeutically effective amount of a compound of this invention. The present invention provides a method of treating a subject suffering from anxiety which comprises administering to the subject a therapeutically effective amount of a compound of this invention. This invention further provides a method of treating a subject suffering from obesity which comprises administering to the subject a therapeutically effective amount of a compound of this invention. In an embodiment of this invention, the subject is a human being.

Furthermore, the present invention is directed to the use of a compound of this invention for the manufacture of a medicament useful for treating depression. Additionally, the present invention is directed to the use of a compound of this invention for the manufacture of a medicament useful for treating anxiety. The present invention further provides for the use of a compound of a compound of this invention for the manufacture of a medicament useful for treating obesity.

The invention will be better understood from the Experimental Details which follow. However, one skilled in the art will readily appreciate that the specific methods and results discussed therein are merely illustrative of the invention as described more fully in the claims which follow thereafter. Furthermore, the variables depicted in Schemes 1-8 are consistent with the variables recited in the Summary of the Invention.

In the Experimental Section, standard acronyms are used. Examples of such acronyms include DMF (N,N-Dimethylformamide); TEA (Triethylamine); DPPA (Diphenylphosphoryl azide); AcOH (Acetic Acid); BzNCS (Benzoylisothiocyanate); BnOH (Benzyl alcohol); MeOH (Methanol); EtMgBr (Ethylmagnesiumbromide); Et₂O (Diethyl Ether); MTBE (Methyl t-butyl ether); EtOAc (Ethyl Acetate); THF (Tetrahydrofuran); rt (room temperature); h (hour); and min (minutes). Furthermore, in certain instances, the methods of preparing the compounds of the invention are described generally by referring to representative reagents such as bases or solvents. The particular reagent identified is representative but is not inclusive and does not limit the invention in any way. For example, representative bases include but are not limited to K₂CO₃, TEA or DIEA (Diisopropylethylamine).

In the Experimental Section, Schemes 4-6 describe the synthesis of α-haloketones that contain a 2-pyridyl or 3-pyridyl group, wherein the 2-pyridyl or 3-pyridyl group is substituted with one or more F, Cl, Br, I or CF₃, and optionally substituted with CH₃.

The term ‘PG’ as defined in Scheme 8 is used to designate a ‘protecting group’. One skilled in the art would be able to select the appropriate protecting group for a particular reaction. Moreover, it may be necessary to incorporate protection and deprotection strategies for substitutents such as amino, amido, carboxylic acid and hydroxyl groups in the synthetic methods described below to synthesize the compounds of Formula I. Methods for protection and deprotection of such groups are well known in the art, and may be found in T. Green, et al., Protection Groups in Organic Synthesis, 1991, 2^{nd} Edition, John Wiley & Sons, New York.

### Experimental Section

General Methods: Anhydrous solvents were purchased from the Aldrich Chemical Company and used as received. The NMR spectra were measured on a Bruker Avance 400 spectrometer and or 300 MHz (Varian) with CDCl₃, DMSO-d₆ or CD₃OD as the solvent. Chemical shifts (6) are expressed in ppm, coupling constants (J) are expressed in Hz, and splitting patterns are described as follows: s=singlet; d=doublet; t=triplet; q=quartet; br=broad; m=multiplet; dd=doublet of doublets; dt=doublet of triplets; td=triplet of doublets; dq=doublet of quartet. Unless otherwise noted, mass spectra were obtained using electrospray ionization (ESMS, Micromass Platform II or Quattro Micro) or Waters ZQ mass spectrometry with Agilent 1100 HPLC system with an autosampler using DAD/UV and Waters ELSD detection system and Inertsil ODS-3 column. For LC-MS determination, two methods were used: Method—I: 8 column, Neutral pH, 20% to 90% Acetonitrile/H₂O with 0.2% Ammonium formate; or Method—II: C18 Column, Acidic pH, 20% to 90% Acetonitrile/H₂O with 0.2% AcOH. Thin-layer chromatography (TLC) was carried out on glass plates pre-coated with silica gel 60 F₂₅₄ (0.25 mm, EM Separations Tech.). Preparative TLC was carried out on glass sheets pre-coated with silica gel GF (2 mm, Analtech). Silica gel column chromatography was performed on Merck silica gel 60 (230-400 mesh).

### List and Source of Chemicals

Most of the reagents used in the experimental section such as 2-fluoro nicotinic acid, 6-trifluoromethyl nicotinic acid, 4-trifluoromethyl nicotinic acid, oxalyl chloride, trimethylsilyldiazomethane, isopropyl sulfonyl chloride, benzoylisothiocyanate, bromine, 48% Hydrobromic acid in AcOH, Diisopropylethyl amine and ethyl magnesium bromide were purchased from the Aldrich Chemical Company. 6-Fluoro-2-methyl nicotinic acid was purchased from Asychem. 2-Fluoronicotinonitrile was purchased from Alfa Aesar. 6-Fluoropicolinic acid and 6-fluoronicotinic acid were purchased from Oakwook Products Inc. and Martrix Scientific.

### PREPARATION OF THE COMPOUNDS OF THE INVENTION

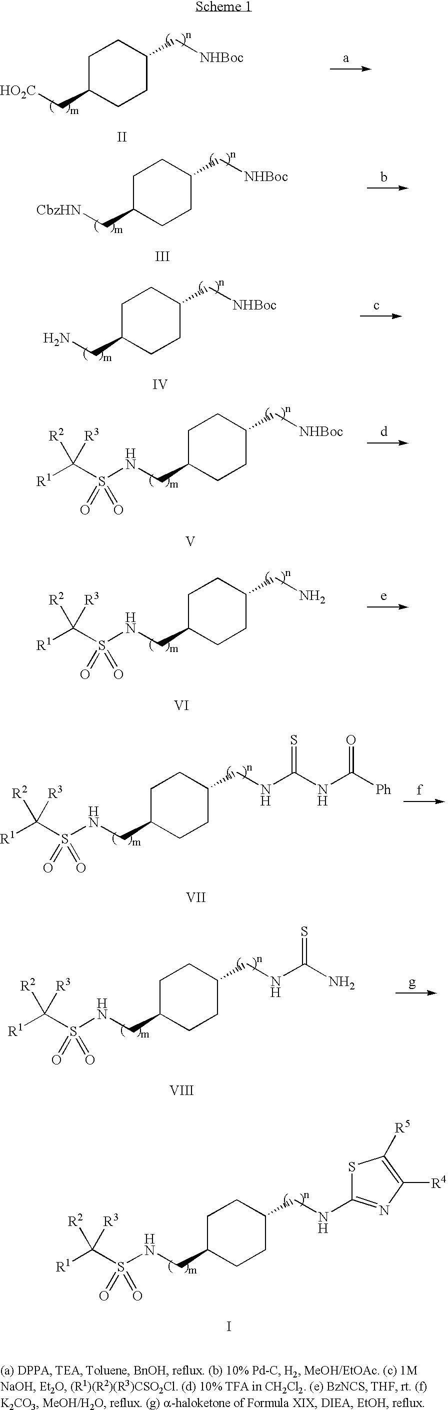

The compounds of Formula I may be synthesized according to the procedures described in Scheme 1. The compounds of Formula II are commercially available or may be synthesized by one skilled in the art. In summary, the carboxylic acids of Formula II are converted to their Cbz-protected amines to afford the compounds of Formula III. The Cbz protecting group is selectively removed to afford the compounds of Formula IV. The resultant amines are coupled with (R¹)(R²)(R³)CSO₂Cl to afford the intermediates of Formula V. The Boc group is removed and the resultant amines of Formula VI are treated with benzoylisothiocyanate to afford the compounds of Formula VII. The compounds of Formula VII are subjected to basic solvolysis to afford the thiourea intermediates of Formula VIII. These intermediates are coupled with the appropriate α-haloketone of Formula XIX to afford the compounds of the invention.

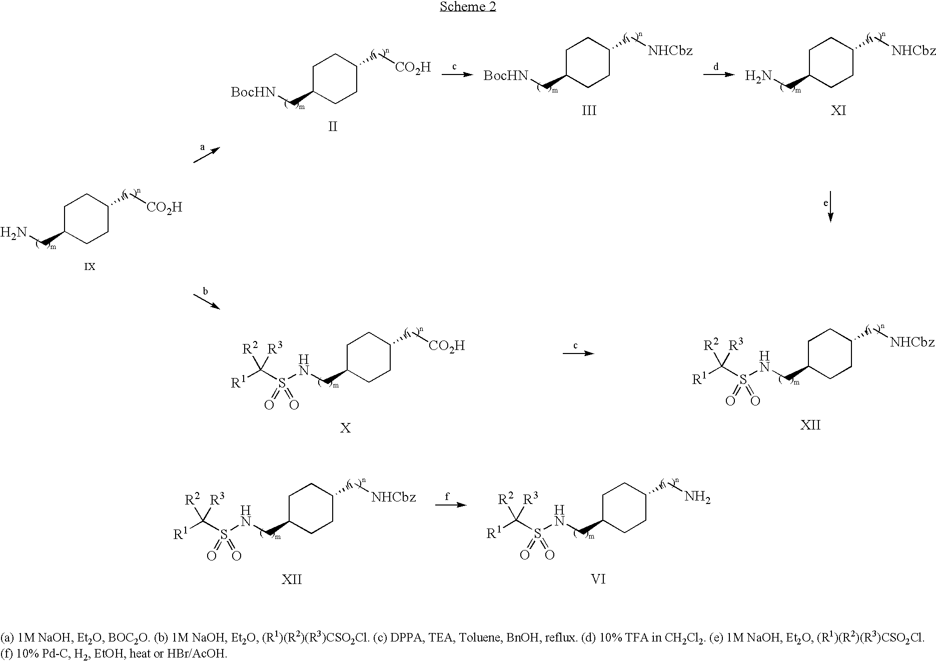

Alternatively, the intermediates of Formula VI may be synthesized according to the procedures outlined in Scheme 2. The starting materials of Formula IX are commercially available or may be synthesized by one skilled in the art. The amino acids of Formula IX may be coupled with (R¹)(R²)(R³)CSO₂Cl to afford the intermediates of Formula X which are further converted to the Cbz-protected amines of Formula XII. Separately, the amino acids of Formula IX may be converted into the mono-Cbz protected intermediates of Formula XI. The amines of Formula XI are coupled with (R¹)(R²)(R³)CSO₂Cl to afford the compounds of Formula XII. The Cbz group of Formula XII is removed to afford the intermediates of Formula VI. Finally, the intermediates of Formula VI may be converted to the compounds of the invention by using the procedures described in Scheme 1.

Additionally, the intermediates of Formula X can be converted to the compounds of Formula V, which are described in Scheme 1, if t-butanol is substituted for benzyl alcohol.

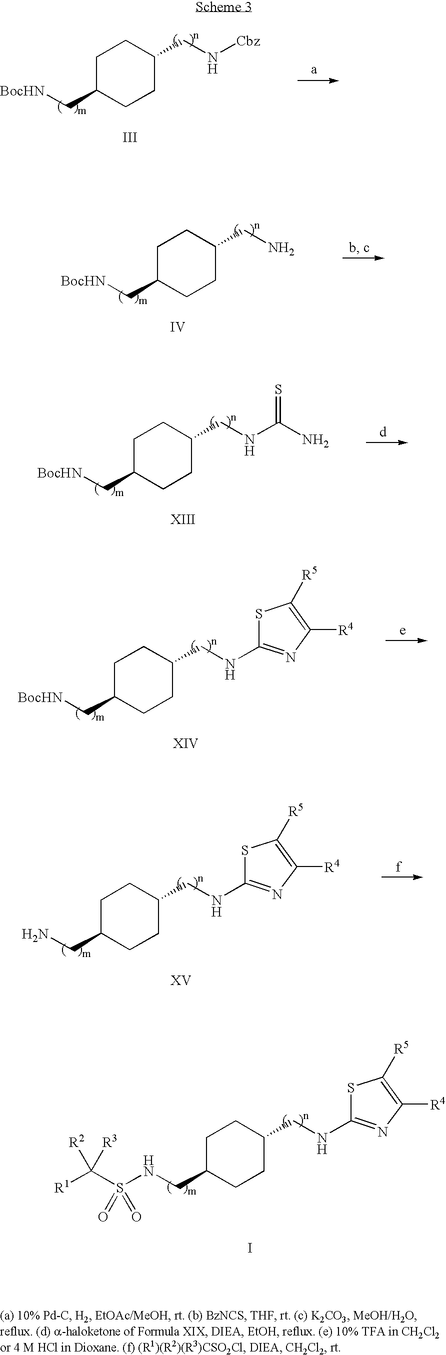

Additionally, the compounds of Formula I may be synthesized according to the procedures outlined in Scheme 3. In summary, the Cbz group of Formula III is removed to afford the intermediates of Formula IV. These intermediates are converted to the thioureas of Formula XIII. The thiazole ring is formed to afford the compounds of Formula XIV. The Boc group is removed to afford the amines of Formula XV. The compounds of the invention are synthesized by coupling of the amines with (R¹)(R²)(R³)CSO₂Cl.

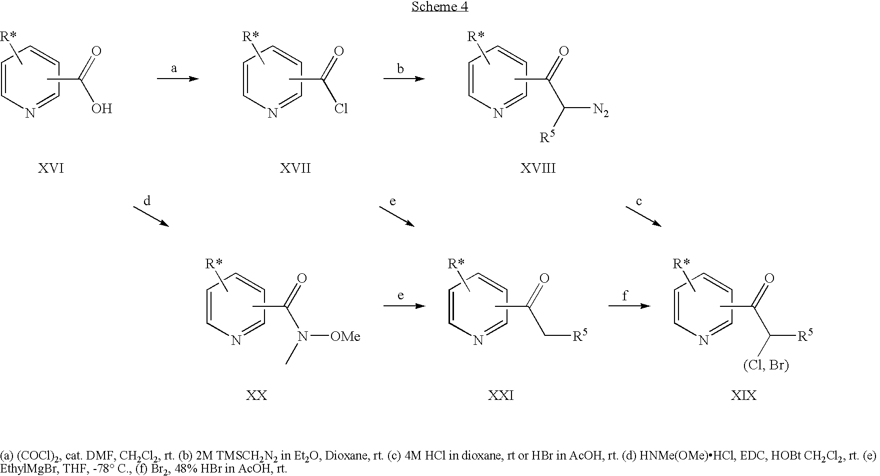

The α-haloketones of Formula XIX may be synthesized according to the procedures described in Scheme 4. The picolinic acids or nicotinic acids of Formula XVI are commercially available or may be synthesized by one skilled in the art. The picolinic acids or nicotinic acids of Formula XVI may be converted to the corresponding acid chlorides of Formula XVII. The acid chlorides may be treated with diazomethane or ethylmagnesiumbromide to afford the intermediates of Formulas XVIII and XXI, respectively. These intermediates may be converted to the α-haloketones of Formula XIX, wherein R⁵ is H or methyl, respectively. Alternatively, the picolinic acids or nicotinic acids of Formula XVI may be converted to the corresponding Weinreb amide of Formula XX which is further converted to the α-haloketones of Formula XIX using ethylmagnesium bromide, wherein R⁵ is methyl. Similarly, if methylmagnesium bromide is substituted for ethylmagnesium bromide, R⁵ is H.

For clarity purposes, the moiety R* is used to denote one or more F, Cl, Br, I or CF₃, and the 2-pyridyl or 3-pyridyl group shown above may optionally be further substituted with methyl.

For representative reaction conditions in connection with the conversion of the carboxylic acids to bromoketones using diazomethane, see A. Gangjee, et al., Bioorg. Med. Chem., 2003, 11, 5155-5170. For representative reaction conditions in connection with the conversion of the haloketones from ketones using Br₂/AcOH or sulfurylchloride and MeOH, see N. Ikemoto, et al., Tetrahedron, 2003, 59, 1317-1325; or using Pyridine-Br₃, HBr/AcOH, see W. C. Patt and M. A. Massa, Tetrahedron Lett., 1997, 38, 1297-1300.

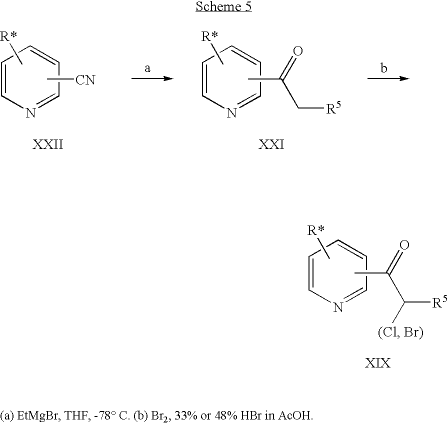

Alternatively, the α-haloketones of Formula XIX may be synthesized according to the procedures described in Scheme 5 from the starting materials of Formula XXII. The cyano compounds are treated with ethylmagnesium bromide to afford the intermediates of Formula XXI, wherein R⁵ is methyl. Similarly, if methylmagnesium bromide is substituted for ethylmagnesium bromide, R⁵ is H. The intermediates of Formula XXI are further converted to the α-haloketones of Formula XIX.

For clarity purposes, the moiety R* is used to denote one or more F, Cl, Br, I or CF₃, and the 2-pyridyl or 3-pyridyl group shown above may optionally be further substituted with methyl.

For representative reaction conditions in connection with the transformation of the cyano group, see N. B. Mehta, J. Clin. Psychiatry, 1983, 44, 56; and S. W. Baldwin and J. E. and Fredericks, Tetrahedron Lett., 1982, 23, 1235-1238.

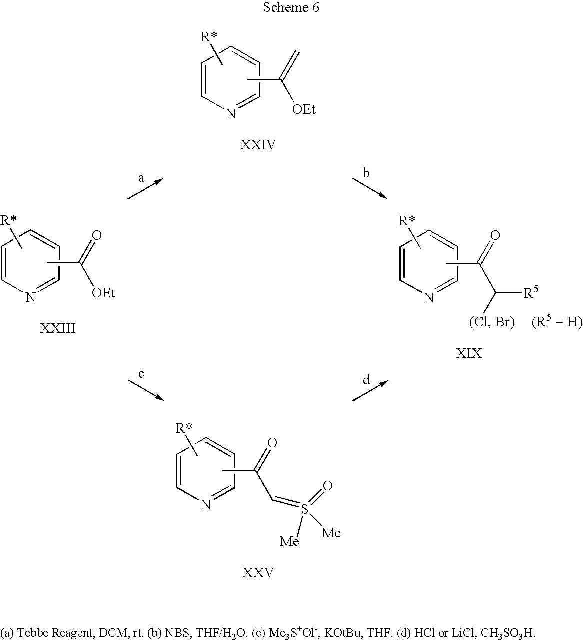

Alternatively, the α-haloketones of Formula XIX wherein R⁵ is H may be synthesized according to the procedures described in Scheme 6. The compounds of Formula XXIII are commercially available or may be synthesized by one skilled in the art. The carboxylate compounds of Formula XXIII are treated with Tebbe reagent to afford the intermediates of Formula XXIV which are converted to the α-haloketones of Formula XIX. Alternatively, the carboxylates of Formula XXIII may be treated with dimethyl sulfonium ylide in the presence of base, such as KOtBu, to afford the intermediates of Formula XXV which are converted to the α-haloketones of Formula XIX.

For clarity purposes, the moiety R* is used to denote one or more F, Cl, Br, I or CF₃, and the 2-pyridyl or 3-pyridyl group shown above may optionally be further substituted with methyl. For representative reaction conditions in connection with the transformation of the esters to the enol ethers using Tebbe reagent, see S. H. Pine, et al., J. Am. Chem. Soc., 1980, 102, 3270. For representative reaction conditions in connection with the transformation of the enol ethers to the α-haloketones, see S. E. Kazzouli, et al., Tetrahedron Lett., 2002, 43, 3193-3196; and F. Bracher, and A. Puzik, Heterocyclic Chem., 2004, 41, 173-176. For representative reaction conditions in connection with the transformation of the esters to the α-haloketones via the dimethylsulfoxonium ylide, see J. E. Baldwin, et al., Synlett, 1993, 51-53; and D. Wang, et al., J. Org. Chem., 2004, 69, 1626-1633.

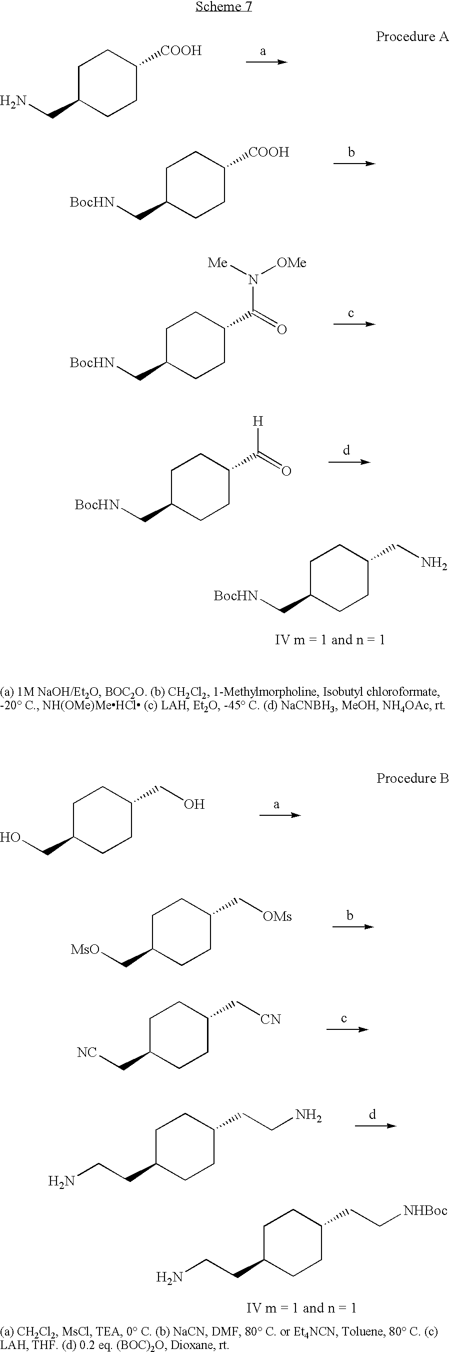

The compounds of Formula IV are used as intermediates in Schemes 1, 2 and 3. These intermediates, wherein n=1 and m=1 (procedure A) or wherein n=2 and m=2, (procedure B) are synthesized according to the procedures described in Scheme 7.

For representative reaction conditions in connection with the synthesis of the diamine in Procedure B see P. Garcia, et al., J. Org. Chem., 1961, 26, 4167-4168. For representative reaction conditions in connection with the synthesis of the mono-protected Boc diamine in Procedure B see J. Hansen, et al., Synthesis, 1982, 5, 404-405 and C. Dardonville, et al., Bioorg. Med. Chem. Lett., 2004, 14, 491-493.

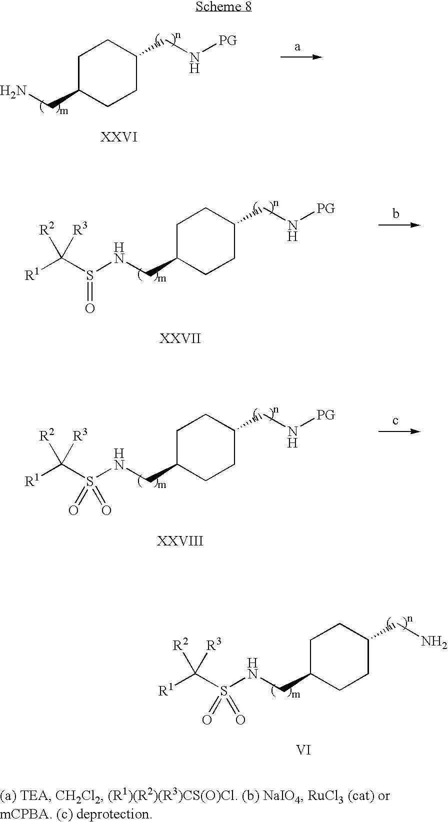

The sulfonamides may also be synthesized according to the procedures outlined in Scheme 8. In summary, the compounds of Formula XXVI are reacted with (R¹)(R²)(R³)CS(O)Cl. to afford the sulfinamides of Formula XXVII. These compounds are oxidized to the intermediates of Formula XXVIII. The protecting group is removed to form the intermediates of Formula VI. Finally, the intermediates of Formula VI may be converted to the compounds of the invention by using the procedures described in Scheme 1.

For representative reaction conditions, see S. Weinreb, J. Org. Chem., 1997, 62, 8604-8608; J. Ellman, Tetrahedron Lett., 2001, 42, 1433-1436; B. Sharpless, Org. Lett., 1999, 1, 783-786; and WO 01/37826.

### Preparation of Intermediates

Representative intermediates were synthesized as follows:

### Intermediate of Formula II

trans-4-{[(tert-butoxy)carbonylamino]methyl}cyclohexanecarboxylic acid: Boc₂O (41.7 g, 190 mmol) was added to a stirred biphasic solution containing trans-4-(aminomethyl)cyclohexanecarboxylic acid (25.0 g, 159 mmol), NaHCO₃ (20.0 g, 238 mmol), water (300 mL) and Et₂O (200 mL) at rt. The pH of the solution was adjusted to pH˜9.0 by adding additional quantities of saturated aqueous NaHCO₃. After stirring for 24 h at rt, the layers were separated and the aqueous layer was acidified to pH 4.0 with 1M aqueous HCl. The aqueous layer was extracted with EtOAc. The organic layer was isolated and washed successively with water and brine. The organic layer was concentrated in vacuo and dried under high vacuum to yield the desired product as a colorless solid (23.3 g, 57%). ¹H NMR (CDCl₃) δ 4.60 (br s, 1H), 2.99 (t, 2H, J=6.4 Hz), 2.29-2.23 (m, 3H), 2.05 (dd, 2H, J=13.6 and 3.2 Hz), 1.84 (dd, 2H, J=13.2 and 2.8 Hz), 1.44 (s, 9H), 1.42 (br m, 1H), 0.97 (dq, 2H, J=25.6, 12.4 and 3.2 Hz).

### Intermediate of Formula III

(tert-butoxy)-N-({trans-4-[(phenylmethoxy)carbonylamino]cyclohexyl}methyl)carboxamide: trans-4-{[(tert-butoxy)carbonylamino]methyl}cyclohexane carboxylic acid (20.3 g, 0.073 mol) was suspended in toluene (420 mL) and chilled to −10° C. in a dry ice bath. DPPA (15.8 mL, 0.073 mol) was added and chilling was continued. TEA (15.3 mL, 0.11 mol) was added drop wise over 10 min. The mixture was removed from the ice bath, warmed to 10° C. and then slowly heated to 70° C. After 15 h, nitrogen evolution was observed to be finished and the solution color turned to yellow. The mixture was cooled to 47° C. and benzyl alcohol (22.8 mL, 0.220 mol) was added. The mixture was heated to 110° C. and stirred overnight. The mixture was cooled to 50° C. and concentrated in vacuo to obtain amber solids. The solids were treated with deionized water (400 mL) and EtOAc (150 mL). The mixture was shaken for 10 min and the layers were isolated. The aqueous layer was extracted several times with EtOAc. The organic layers were combined, dried and concentrated in vacuo. The resultant solid was triturated in MTBE to afford a white solid (17.8 g, 67%) as the desired product. ¹H NMR (CDCl₃) δ 7.34-7.286 (m, 3H), 7.13 (d, 2H, J=8.0 Hz), 6.78 (t, 1H, J=6.0 Hz), 4.97 (s, 2H), 3.3-3.14 (br, m, 1H), 2.73 (t, 1H, J=6.3 Hz), 1.78 (d, 2H, J=10.6 Hz), 1.64 (d, 2H, J=11.8 Hz), 1.35 (s, 6H), 1.35-1.00 (m, 3H), 0.87 (q, 2H, J=12.5 and 2.8 Hz).

### Intermediate of Formula IV

N-[(trans-4-aminocyclohexyl)methyl](tert-butoxy)carboxamide: 10% Pd—C (0.20 g) was added to a stirred solution of (tert-butoxy)-N-({trans-4-[(phenylmethoxy)carbonylamino]cyclohexyl}methyl)carboxamide (2.0 g, 5.5 mmol) in EtOAc/ MeOH (1:1, 50 mL) at rt. The mixture was degassed and purged with hydrogen twice, and further stirred at rt under atmospheric pressure of hydrogen for 2 h. The mixture was filtered through celite and the filter cake was washed with EtOAc. The filtrate was concentrated in vacuo to afford the desired compound as a solid (1.3 g, 91%). ¹H NMR (CDCl₃) δ 4.59 (br s, 1H), 2.97 (t, 2H, J=6.4 Hz), 2.63-2.58 (m, 1H), 1.87 (d, 2H, J=12.4 Hz), 1.75 (d, 2H, J=12.4 Hz), 1.44 (s, 9H), 1.37 (m, 1H), 1.12-0.96 (m, 4H). ESMS m/e: 173 ((M+H)−55)⁺.

### Intermediate of Formula V

(tert-butoxy)-N-[(trans-4-{[(methylethyl)sulfonyl]amino}cyclohexyl)methyl]carboxamide: Isopropyl sulfonyl chloride (6.2 mL, 7.9 g, 56 mmol) was added drop wise at rt to a stirred biphasic solution containing N-[(trans-4-aminocyclohexyl)methyl](tert-butoxy)carboxamide (10 g, 44 mmol), 1M aqueous NaOH (100 mL) and Et₂O (100 mL). After stirring for 2 h, a white precipitate appeared. The precipitate was collected by filtration, washed with Et₂O and dried in vacuo to obtain the desired product as a solid (9.5 g, 65%). ¹H NMR (CDCl₃) δ 4.58 (br s, 1H), 3.89 (d, 1H, J=8.0 Hz), 3.23 (septet, 1H, J=4.4 Hz), 2.96 (t, 2H, J=6.4 Hz), 2.61 (m, 1H), 2.09 (d, 2H, J=11.6 Hz), 1.89-1.74 (m, 3H), 1.44 (s, 9H), 1.37 (d, 6H, J=6.8 Hz), 1.22 (dq, 1H, J=13.2 and 3.6 Hz), 1.09-1.00 (br m, 3H). ESMS m/e: 279 ((M+H)−55)⁺.

### Intermediate of Formula VI

[trans-4-(aminomethyl)cyclohexyl][(methylethyl)sulfonyl]amine: TFA (5 mL) was added at rt to a stirred solution containing (tert-butoxy)-N-[(trans-4-{[(methylethyl)sulfonyl]amino}cyclohexyl)methyl]carboxamide (1.3 g, 3.9 mmol) and CH₂Cl₂ (45 mL). After 4 h, the solution was concentrated in vacuo and the residue was re-dissolved in CHCl₃. The CHCl₃ solution was washed successively with 1M aqueous NaOH and brine, dried over Na₂SO₄ and then concentrated in vacuo to provide the free base as a solid (0.88 g, 97%). ¹H NMR (CDCl₃) δ 3.19-3.09 (m, 2H), 2.82 (dd, 2H, J=13.6 and 7.2 Hz), 2.07 (d, 2H, J=13.6 Hz), 1.88 (d, 2H, J=6.0 Hz), 1.64-1.60 (br m, 2H), 1.51-1.30 (m, 2H), 1.34 (d, 6H, J=6.8 Hz), 1.23-1.08 (m, 3H). ESMS m/e: 235 (M+H)⁺.

### Intermediate of Formula VII

N-({[(trans-4-{[(methylethyl)sulfonyl]amino}cyclohexyl)methyl]amino}thioxomethyl)benzamide: Benzoylisothiocyanate (1.6 g, 10 mmol) was added to a solution of [trans-4-(aminomethyl)cyclohexyl][(methylethyl)sulfonyl]amine (2.3 g, 10 mmol) in THF (100 mL) under an argon atmosphere and then stirred at rt overnight. The reaction mixture was concentrated in vacuo and the resultant gum-like material was triturated with Hexanes to obtain the product as a pale yellow solid. (3.8 g, 96%). ¹H NMR (CDCl₃) δ 10.86 (s, 1H), 9.08 (s, 1H), 7.86 (d, 1H, J=4.0 Hz), 7.64 (dt, 1H, J=5.6 and 1.2 Hz), 7.54-7.49 (m, 3H), 4.18 (d, 1H, J=8.4 Hz), 3.59 (t, 2H, J=6.0 Hz), 3.25 (septet, 1H, J=4.0 Hz), 2.14 (dd, 2H, J=7.2 and 2.4 Hz), 1.91 (d, 2H, J=6.4 Hz), 1.74 (m, 1H), 1.29 (d, 6H, J=12.8 Hz), 1.25-1.12 (m, 2H). ESMS m/e: 398 (M+H)⁺.

### Intermediate of Formula VIII

(trans-4-{[(aminothioxomethyl)amino]methyl}cyclohexyl)[(methylethyl)sulfonyl]amine: K₂CO₃ (2.00 g, 14.5 mmol) was added to a solution containing N-({[(trans-4-{[(methylethyl)sulfonyl]amino}cyclohexyl)methyl]amino}thioxomethyl)benzamide (3.80 g, 9.57 mmol), MeOH (75 mL) and H₂O (25 mL). The resulting turbid mixture was refluxed for 16 h. Note that upon refluxing the mixture turned into a clear homogenous solution. The solution was allowed to cool and concentrated in vacuo to yield a solid. This solid was dissolved in acetone and filtered through a pad of celite, followed by additional washings of the celite with acetone. The filtrate was concentrated in vacuo to afford the desired product as a pale yellow solid (2.20 g, 79%). ¹H NMR (CDCl₃) δ 6.98 (br s, 1H), 6.39-6.32 (br s, 2H), 3.50 (br s, 1H), 3.32 (m, 1H), 3.14 (m, 2H), 2.16-2.13 (br m, 1H), 1.94-1.92 (m, 2H), 1.82-1.79 (m, 2H), 1.52-1.36 (m, 2H), 1.38 (d, 6H, J=6.8 Hz), 1.26-1.10 (m, 2H). ESMS m/e: 294 (M+H)⁺.

### Intermediate of Formula X

trans-4-({[(methylethyl)sulfonyl]amino}methyl)cyclohexanecarboxylic acid: Isopropyl sulfonyl chloride (10.9 g, 77.0 mmol) was added drop wise to a solution of trans-4-(aminomethyl)cyclohexanecarboxylic acid (10.0 g, 63.7 mmol) in 1M aqueous NaOH (150 mL, 150 mmol), cooled in an ice bath. The solution was stirred for 24 h and then acidified to pH˜4 with 2 M aqueous HCl. The solids were collected by filtration and dried under vacuum at rt to afford the desired product as a white solid (5.0 g, 33%). ¹H NMR (CDCl₃) δ 4.91 (br s, 1H), 3.20 (septet, 1H, J=6.8 Hz), 2.92 (d, 2H, J=6.8 Hz), 2.24 (tt, 1H, J=12.4 and 3.6 Hz), 2.03 (dd, 2H, J=10.4 and 3.2 Hz), 1.92 (dd, 2H, J=10.8 and 3.2 Hz), 1.48-1.41 (m, 3H), 1.34 (d, 6H, J=6.8 Hz), 1.01 (dq, 2H, J=25.2, 13.2 and 3.6 Hz). ESMS m/e: 264 (M+H)⁺.

### Intermediate of Formula XI

N-[trans-4-(aminomethyl)cyclohexyl](phenylmethoxy)carboxamide: (tert-butoxy)-N-({trans-4-[(phenylmethoxy)carbonylamino]cyclohexyl}methyl)carboxamide (4 g, 11 mmol) was treated with 25% TFA in CH₂Cl₂ (50 mL). After stirring 5 h, the solvents were removed in vacuo. The crude product was redissolved in CH₂Cl₂ and washed with saturated aqueous NaHCO₃ and brine. The organic layer was isolated, dried over Na₂SO₄ and concentrated in vacuo to afford the desired product as an oil (quantitative yield). ¹H NMR (DMSO-d) δ 7.18-7.03 (m, 5H), 4.78 (s, 2H), 3.06-2.96 (m, 1H), 2.35 (d, 2H, J=6.8 Hz), 1.62-1.48 (m, 4H), 1.20-1.11 (m, 1H), 0.97-0.69 (m, 4H). ESMS m/e: 263 (M+H)⁺.

### Intermediate of Formula XII

N-[trans-4-({[(methylethyl)sulfonyl]amino}methyl)cyclohexyl](phenylmethoxy)carboxamide: Isopropyl sulfonyl chloride (6.6 g, 46 mmol) was added drop wise to a solution containing N-[trans-4-(aminomethyl)cyclohexyl](phenylmethoxy)carboxamide (11 g, 42 mmol), TEA (7.0 g, 70 mmol) in anhydrous CH₂Cl₂ (150 mL) at 0° C. The reaction was allowed to warm to rt and stirred overnight. The solution was washed with saturated aqueous NaHCO₃, dried over Na₂SO₄ and concentrated in vacuo. The crude product was purified by silica gel column chromatography using an increasing gradient of EtOAc in Hexanes. The fractions containing the product were combined and concentrated in vacuo. Recrystallization from EtOH gave the desired product (5.0 g, 32%). ¹H NMR (CDCl₃) δ 7.38-7.31 (m, 5H), 5.08 (s, 2H), 4.66-4.57 (br m, 1H), 4.13-4.06 (br m, 1H), 3.49-3.4 (br m, 1H), 3.19-3.11 (m, 1H), 2.97 (t, 2H, J=6.7 Hz), 2.08-2.05 (br m, 2H), 1.86-1.83 (br m, 2H), 1.50-1.40 (m, 1H), 1.36 (d, 6H, J=0.9 Hz), 1.18-0.99 (m, 4H). ESMS m/e: 369 (M+H)⁺.

### Intermediate of Formula XII to Intermediate of Formula VI

[(trans-4-aminocyclohexyl)methyl][(methylethyl)sulfonyl]amine: N-[trans-4-({[(methylethyl)sulfonyl]amino}methyl)cyclohexyl](phenylmethoxy)carboxamide (62 g, 0.168 mole) and 10% Pd—C (12 g) in anhydrous EtOH (600 mL) was shaken under hydrogen at 55 psi at 60° C. for 6 h. The reaction mixture was filtered through celite and celite was washed with EtOH. The combined EtOH filtrate was concentrated in vacuo. Toluene (200 mL) was added and evaporated to obtain the desired product as a white solid (quantitative yield). ¹H NMR (CDCl₃) δ 3.60 (br s, 2H), 3.06 (quintet, 1H), 2.69 (d, 2H, J=11.2 Hz), 2.58 (m, 1H), 1.79 (d, 2H, J=11.2 Hz), 1.68 (d, 2H, J=11.2 Hz), 1.24 (br m, 1H), 1.14 (d, 6H, J=11.2 Hz), 1.09 (2H, q, J=22.5 and 9.0 Hz) and 0.86 (2H, q, J=22.5 and 9.0 Hz).

### Intermediate of Formula XIII

N-({trans-4-[(aminothioxomethyl)amino]cyclohexyl}methyl)(tert-butoxy)carboxamide: Benzoylisothiocyanate (0.755 g, 4.63 mmol) was added to a stirred solution of (tert-butoxy)carboxamidemethylcyclohexyl-trans-4-amine (1.2 g, 4.6 mmol) in THF (50 mL) at rt under argon atmosphere. After stirring for 24 h, the solution was concentrated in vacuo to afford a viscous material. Trituration of the viscous material with Hexanes afforded N-({trans-4-[(aminothioxomethyl)amino]cyclohexyl}methyl) (tert-butoxy)carboxamidebenzamide as a pale yellow color solid (1.71 g, 90% yield). ¹H NMR (CDCl₃) δ 10.61 (d, 1H, J=7.2 Hz), 8.89 (s, 1H), 7.82 (d, 1H, J=7.6 Hz), 7.63 (t, 1H, J=7.2 Hz), 7.54-7.49 (m, 3H), 4.61 (br s, 1H), 4.24-4.19 (m, 1H), 3.02 (t, 2H, J=6.0 Hz), 2.27 (dd, 2H, J=7.6 and 2.0 Hz), 1.86 (d, 2H, J=11.6 Hz), 1.45 (s, 10H), 1.32 (dq, 2H, J=24.8, 12.4 and 3.2 Hz), 1.28 (dq, 2H, J=25.6, 13.2 and 2.4 Hz). ESMS m/e: 336 ((M+H)−55)⁺.

N-({trans-4-[(aminothioxomethyl)amino]cyclohexyl}methyl)(tert-butoxy)carboxamidebenzamide (1.65 g, 4.0 mmol) was dissolved in MeOH (25 mL). Water (5 mL) was added followed by the addition of K₂CO₃ (1.66 g, 12.0 mmol). The resultant mixture was allowed to reflux overnight. After refluxing for 16 h, the solvents were removed in vacuo and the resultant solid was dried under high vacuum. The solid was re-dissolved in acetone and filtered through a pad of celite. Concentration of the filtrate in vacuo afforded the desired product as a pale yellow color solid (1.1 g). ¹H NMR (CDCl₃) δ 6.17 (br s, 1H), 4.85 (br s, 1H), 2.99 (t, 2H, J=7.2 Hz), 2.09 (d, 2H, J=12.0 Hz), 1.83 (d, 2H, J=12.0 Hz), 1.44 (s, 10H), 1.29-0.78 (m, 4H). ESMS m/e: 288 (M+H)⁺.

### Intermediate of Formula XIV

### (tert-butoxy)-N-({4-[(trans-4-(6-fluoro-pyridin-2-yl)(1,3-thiazol-2-yl))amino]cyclohexyl}methyl)carboxamide

DIEA (1.17 mL, 6.45 mmol) was added to a solution of 2-bromo-1-(6-fluoro-pyridin-2-yl)ethan-1-one HCl salt (450 mg, 2.18 mmol) in anhydrous EtOH (5 mL) under an argon atmosphere. After stirring for 5 min at rt, N-({trans-4-[(aminothioxomethyl)amino]cyclohexyl}methyl)(tert-butoxy)carboxamide (627 mg, 2.18 mmol) was added and the reaction was refluxed overnight. EtOH was removed in vacuo and the residue was re-dissolved in CH₂Cl₂. The organic layer was washed with water, brine, dried over Na₂SO₄ and concentrated in vacuo. The crude product was purified by silica gel column chromatography, eluting with 60% EtOAc in Hexanes to yield the desired product (425 mg, 48%) as a yellow colored solid. ¹H NMR (CDCl₃) δ 7.81-7.78 (m, 2H), 7.31 (s, 1H), 6.81-6.78 (m, 1H), 5.04 (d, 1H, J=8 Hz), 4.60 (br s, 1H), 3.36-3.30 (m, 1H), 3.02 (t, 2H, J=6.4 Hz), 2.25 (d, 2H, J=12.0 Hz), 1.86 (d, 2H, J=12.4 Hz), 1.49-1.30 (br m, 1H), 1.45 (s, 9H), 1.28-1.22 (m, 2H), 1.11 (q, 2H, J=25.0 and 12.0 Hz). ESMS m/e: 407 (M+H)⁺.

### Intermediate of Formula XV

### [trans-4-(aminomethyl)cyclohexyl](4-(6-fluoro-pyridin-2-yl)(1,3-thiazol-2-yl))amine

(tert-butoxy)-N-({trans-4-[(4-(2-pyridyl)(1,3-thiazol-2-yl))amino]cyclohexyl}methyl)carboxamide (300 mg, 0.74 mmol) was treated with 4 M HCl in dioxane (15 mL) in CH₂Cl₂ (10 mL) under argon atmosphere. The reaction mixture was stirred at rt overnight and the product precipitated from the reaction mixture. The resulting precipitate was collected by filtration as the hydrochloride salt (280 mg, 92%). ¹H NMR (CD₃OD) δ 8.14-8.07 (m, 1H), 7.89 (d, 1H, J=8.0 Hz), 7.18 (d, 1H, J=8.0 Hz), 3.66 (br m, 1H), 2.88 (d, 2H, J=7.2 Hz), 2.25 (d, 2H, J=12.0 Hz), 2.01 (d, 2H, J=12.0 Hz), 1.77-1.73 (br m, 1H), 1.59 (q, 2H, J=24.0 and 12.0 Hz), 1.29 (q, 2H, J=24.0 and 12.0 Hz). ESMS m/e: 307 (M+H)⁺.

### Intermediate of Formula IV (wherein m=n=1)

Representative compounds as described in Scheme 7 were synthesized as follows: (trans-4-{[(tert-butoxy)carbonylamino]methyl}cyclohexyl)-N-methoxy-N-methylcarboxamide: 1-Methylmorpholine (5.0 g, 49.6 mmol) was added to a stirred solution of trans-4-{[(tert-butoxy)carbonylamino]methyl}cyclohexanecarboxylic acid (8.5 g, 33 mmol) in CH₂Cl₂ (100 mL). The solution was cooled to −20° C. and isobutyl chloroformate (5.9 g, 43.0 mmol) was added drop wise over a period of 10 min. The resulting pale yellow color solution was allowed to warm to rt and then stirred for an additional hour. The solution was cooled to −20° C. and 1-Methylmorpholine (5.0 g, 50 mmol) was added followed by the addition of a solution of N,O-dimethylhydroxylamine (3.9 g, 40 mmol) in CH₂Cl₂. After the addition was complete, the solution was warned to rt and was stirred overnight. The reaction was quenched with water and washed successively with aqueous citric acid, water, and brine. The CH₂Cl₂ layer was concentrated in vacuo and the resulting material was purified by silica gel column chromatography (30% EtOAc in Hexanes) to afford the desired product as a colorless viscous material (7.8 g, 79%). ¹H NMR (CDCl₃) δ 4.67 (br s, 1H), 3.69 (s, 3H), 3.18 (s, 3H), 2.99 (t, 2H, J=6.4 Hz), 2.65 (br m, 1H, J=9.6 Hz), 1.83 (d, 2H, J=10.0 Hz), 1.53-1.49 (m, 3H), 1.44 (s, 9H), 1.0-0.91 (m, 4H). ESMS m/e: 300 (M+H)⁺.

(tert-butoxy)-N-[(trans-4-formylcyclohexyl)methyl]carboxamide: A solution of 1M LAH in THF (24.7 mL, 24.7 mmol) was added over a period of 15 min to a stirred solution of (trans-4-{[(tert-butoxy)carbonylamino]methyl}cyclohexyl)-N-methoxy-N-methylcarboxamide (7.4 g, 24.7 mmol) in Et₂O (200 mL) under argon atmosphere at −78° C. After the addition was complete, the reaction was stirred for an additional 15 min then allowed to warm to 0° C. for 1 h and then re-cooled to −78° C. The reaction was quenched with 1N aqueous KHSO₄ and filtered over celite containing Na₂SO₄. The celite was washed with Et₂O and the filtrate was concentrated in vacuo to afford the desired product as a viscous gum (6.0 g, >99%). ¹H NMR (CDCl₃) δ 9.62 (s, 1H), 4.67 (br s, 1H), 3.00 (t, 2H, J=6.0 Hz), 2.19 (t, 1H, J=8.8 Hz), 2.03 (d, 2H, J=13.2 Hz), 1.89 (d, 2H, J=13.2 Hz), 1.44 (s, 9H), 1.27 (dq, 2H, J=25.6, 13.2 and 3.6 Hz), 1.05-0.95 (m, 2H). ESMS m/e: 186 ((M+H)−55)⁺.

N-{[trans-4-(aminomethyl)cyclohexyl]methyl}(tert-butoxy)carboxamide: NaCNBH₃ (2.4 g, 38 mmol) was added to a stirred solution containing the (tert-butoxy)-N-[(trans-4-formylcyclohexyl)methyl]carboxamide (6.0 g, 25 mmol), ammonium acetate (29 g, 374 mmol) and MeOH (100 mL) under argon atmosphere at rt. The solution was stirred for 18 h at rt and concentrated in vacuo. The crude product was purified by silica gel flash column chromatography to yield the desired amine (1.5 g, 26%). ¹H NMR (CDCl₃) δ 4.64 (br s, 1H), 2.97 (t, 2H, J=6.4 Hz), 2.53 (d, 1H, J=6.4 Hz), 2.41 (d, 1H, J=6.4 Hz), 1.79 (q, 2H, J=19.6 and 9.2 Hz), 1.44 (s, 9H), 1.50-1.30 (br m, 4H), 1.0-0.81 (m, 4H). ESMS m/e: 242 (M+H)⁺.

### Intermediate of Formula XVII

6-Fluoro-pyridine-2-carbonyl chloride: Oxalyl chloride (1.35 g, 10.6 mmol) was added to a stirred solution of 6-fluoropicolinic acid (1.0 g, 7.09 mmol) in CH₂Cl₂ (25 mL) at rt under argon atmosphere. A drop of DMF was added to initiate reaction. After stirring for 24 h at rt, the solvents were removed in vacuo to yield the acid chloride as a colorless solid (1.35 g, 97%).

### Intermediate of Formula XIX (Wherein R⁵ is H)

2-Chloro-1-(6-fluoro-pyridin-2-yl)-ethanone: 6-Fluoro-pyridine-2-carbonyl chloride (1.35 g, 6.90 mmol) was dissolved in dioxane (25 mL) and cooled to 0° C. A 2M ethereal solution of trimethylsilyidiazomethane (6.90 mL, 1.57 g, 13.82 mmol) was added drop wise and the reaction was stirred at rt for 24 h to generate the intermediate of Formula XVIII. 4M HCl in dioxane (10 mL) was added to the solution and stirred for 2 h at rt. The solution was concentrated in vacuo to afford the title compound as a dark brown colored solid (1.45 g, >99%). ¹H NMR (CDCl₃) δ 8.05-7.99 (m, 3H), 7.24-7.20 (m, 1H), 5.02 (s, 2H). ESMS m/e: 174 (M+H)⁺.

The following compounds were prepared analogously:

2-Chloro-1-(2-fluoro-pyridin-3-yl)-ethanone: ¹H NMR (CDCl₃) δ: 9.26 (s, 1H), 8.96 (d, 1H, J=8.4 Hz), 8.06 (d, 1H, J=8.4 Hz), 4.87 (s, 2H). ESMS m/e: 174 (M+H)⁺.

2-Chloro-1-(6-fluoro-2-methyl-pyridin-3-yl)-ethanone: ¹H NMR (CDCl₃) δ 8.39 (t, 1H, J=8.04 Hz), 7.01 (d, 1H, J=5.8 Hz), 4.87 (s, 2H), 2.61 (s, 3H). ESMS m/e: 188 (M+H)⁺.

### Intermediate of Formula XX

6-Fluoro-N-methoxy-N-methyl-nicotinamide: DIEA (5.0 mL, 3.63 g, 28.4 mmol) was added to a stirred solution of 2-fluoro-5-pyridine carboxylic acid (2.0 g, 14.2 mmol) and N,O-dimethyl-hydroxylamine hydrochloride (1.66 g, 17.0 mmol) in CH₂Cl₂ (30 mL) at rt. Diethyl cyanophosphonate (4.25 mL, 28.4 mmol) was added to the solution with slight warming. After stirring for 18 h at rt, the solution was diluted with CH₂Cl₂ and carefully washed with saturated aqueous NaHCO₃. The organic layer was separated, dried over anhydrous Na₂SO₄ and concentrated in vacuo. The product was purified by silica gel flash chromatography (50% EtOAc in Hexanes) to furnish the title product as an oil (2.58 g, 99%). ¹H NMR (CDCl₃) δ 8.62 (s, 1H), 8.19 (t, 1H, J=5.3 Hz), 6.95 (d, 1H, J=4.0 Hz), 3.59 (s, 3H), 3.40 (s, 3H). ESMS m/e: 185 (M+H⁺).

The following compound was prepared analogously:

2-Fluoro-N-methoxy-N-methyl-nicotinamide: ¹H NMR (CDCl₃) δ 8.29 (d, 1H, J=3.8 Hz), 7.91 (t, 1H, J=5.3 Hz), 7.22 (m, 1H), 3.58 (s, 3H), 3.35 (s, 3H). ESMS m/e: 185 (M+H)⁺.

### Intermediate of Formula XXI

1-(6-Fluoro-pyridin-3-yl)-propan-1-one: A 3M solution of ethylmagnesium bromide in Et₂O (3.26 mL, 9.8 mmol) was added to a stirred solution of 6-fluoro-N-methoxy-N-methyl-nicotinamide (2.0 g, 6.53 mmol) in THF (20 mL) at −78° C. After stirring for 2 h at −78° C., the reaction was quenched by the addition of aqueous citric acid (10% w/v, 1 mL). The reaction mixture was warmed to rt and stirred for an additional 18 h.

The organic layer was separated, dried over anhydrous Na₂SO₄ and concentrated in vacuo. The product was purified by silica gel flash chromatography (35% EtOAc in Hexanes) to furnish the title compound as an oil (0.47 g, 50%). ¹H NMR (CDCl₃) [symbol] 8.82 (s, 1H), 8.39 (t, 1H, J=5.4 Hz), 7.00 (d, 1H, J=4.1 Hz), 3.01 (q, 2H, J=7.4 Hz), 1.21 (t, 3H, J=7.2 Hz). ESMS m/e: 154 (M+H)⁺.

The following compounds were prepared analogously:

1-(2-Fluoro-pyridine-3-yl)-propan-1-one: ¹H NMR (CDCl₃) δ 8.29-8.39 (m, 2H), 7.32 (m, 1H), 3.05 (q, 2H, J=7.4 Hz), 1.21 (t, 3H, J=7.2 Hz). ESMS m/e: 154 (M+H)⁺.

1-(6-Trifluoromethyl-pyridin-3-yl)-propan-1-one: ¹H NMR (CDCl₃) δ 8.89 (d, 1H, J=1.2 Hz), 8.79 (s, 1H), 7.62 (d, 1H, J=5.1 Hz), 2.92 (q, 2H, J=14.4 and 7.2 Hz), 1.25 (t, 3H, J=7.2 Hz). ESMS m/e: 204 (M+H)⁺.

### Intermediate of Formula XIX (Wherein R⁵ is CH₃)

2-Bromo-1-(6-fluoro-pyridin-3-yl)-propan-1-one HBr salt: 1-(6-Fluoro-pyridin-3-yl)-propan-1-one (0.47 g, 3.10 mmol) was dissolved in AcOH (4 mL). 33% HBr in AcOH was added (0.54 mL, 3.10 mmol) to the solution with vigorous stirring. Bromine (0.16 mL, 3.10 mmol) was added to the solution and stirred at rt for 1.5 h. The orange solution was concentrated in vacuo to give the title compound as an orange semi solid. (1.13 g, >99%). ESMS m/e: 233 (M+H)⁺.

The following compounds were prepared analogously:

2-Bromo-1-(2-fluoro-pyridin-3-yl)-propan-1-one. ESMS m/e: 233 (M+H)⁺.

2-Bromo-1-(6-trifluoromethyl-pyridin-3-yl)-propan-1-one. ESMS m/e: 281 (M+H)⁺.

### COMPOUNDS OF THE INVENTION

The following compounds of the invention were prepared from intermediate Formula VIII and synthesized according to the procedures described in Scheme 1:

### EXAMPLE 1a [(Methylethyl)sulfonyl](trans-4-{[(4-(6-fluoro-pyridin-2-yl)(1,3-thiazol-2-yl))amino]methyl}cyclohexyl)amine

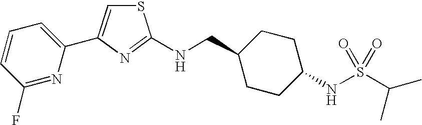

N-({[(trans-4-{[(methylethyl)sulfonyl]amino}cyclohexyl)methyl]amino}thioxomethyl) amide (0.10 g, 0.34 mmol) was added to a stirred solution of 2-chloro-1-(6-fluoro-pyridin-2-yl)-ethanone (0.075 g, 0.34 mmol) in EtOH (10 mL) at rt. DIEA (0.18 mL, 0.132 g, 1.02 mmol) was added to the solution. The reaction mixture was heated at reflux for 4 h, cooled to rt and concentrated in vacuo. The resulting residue was re-dissolved in CHCl₃ and washed successively with aqueous citric acid, water and brine. The organic layer was isolated, dried over anhydrous Na₂SO₄, and concentrated in vacuo. The product was purified by preparative TLC (70% EtOAc in Hexanes) to furnish the title compound as a colorless foam (0.112 g, 76%). ¹H NMR (CDCl₃) δ 7.81-7.76 (m, 2H), 7.30 (s, 1H), 6.79 (dd, 1H, J=5.7 and 2.7 Hz), 5.59 (t, 1H, J=5.1 Hz), 4.29 (d, 1H, J=8.4 Hz), 3.27-3.20 (m, 1H), 3.15 (septet, 1H, J=6.6 Hz), 3.12 (t, 2H, J=7.5 Hz), 2.09 (d, 2H, J=11.7 Hz), 1.88 (d, 2H, J=12.6 Hz), 1.60-1.54 (br m, 1H), 1.37 (d, 6H, J=6.9 Hz), 1.25-1.06 (m, 4H). LC-MS m/e: 413 (M+H)⁺; t_{R}=1.29. (Method—II).

The following compounds were prepared analogously:

### EXAMPLE 1b [(Methylethyl)sulfonyl]({trans-4-[(4-(6-fluoro-pyridin-2-yl)(1,3-thiazol-2-yl))amino]cyclohexyl}methyl)amine

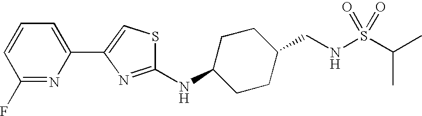

Prepared from N-({[(trans-4-{[(methylethyl)sulfonyl]methylamino}cyclohexyl]amino}thioxomethyl)amide and 2-chloro-1-(6-fluoro-pyridin-2-yl)-ethanone.

Yield: (87%). LC-MS m/e: 413 (M+H)⁺; t_{R}=1.26. (Method—II).

### EXAMPLE 1c [(Methylethyl)sulfonyl]({trans-4-[(4-(2-methyl,6-fluoro-pyridin-3-yl)(1,3-thiazol-2-yl))amino]cyclohexyl}methyl)amine

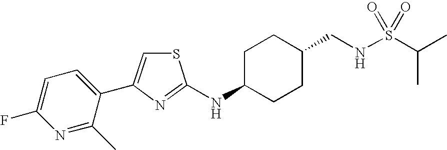

Prepared from N-({[(trans-4-{[(methylethyl)sulfonyl]methylamino}cyclohexyl]amino}thioxomethyl)amide and 2-chloro-1-(6-fluoro-2-methyl-pyridin-3-yl)-ethanone.

Yield: 97%. LC-MS m/e: 427 (M+H)⁺; t_{R}=1.15. (Method—II).

### EXAMPLE 1d [(Methylethyl)sulfonyl](trans-4-({[(4-(2-fluoro-pyridin-3-yl)(1,3-thiazol-2-yl))amino]methyl}cyclohexyl)amine

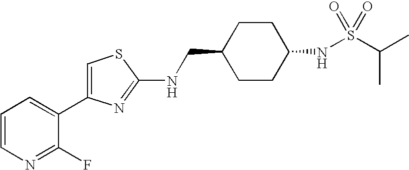

Prepared from N-({[(trans-4-{[(methylethyl)sulfonyl]amino}cyclohexyl)methyl]amino}thioxomethyl)amide and 2-chloro-1-(2-fluoro-pyridin-3-yl)-ethanone.

Yield: 56%. LC-MS m/e: 413 (M+H)⁺; t_{R}=1.23. (Method—I).

### EXAMPLE 1e [(Methylethyl)sulfonyl]({trans-4-[(4-(2-fluoro-pyridin-3-yl)(1,3-thiazol-2-yl))amino]cyclohexyl}methyl)amine

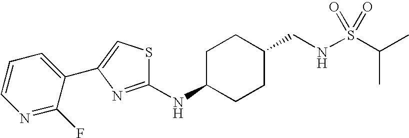

Prepared from N-({[(trans-4-{[(methylethyl)sulfonyl]methylamino}cyclohexyl]amino}thioxomethyl)amide and 2-chloro-1-(2-fluoro-pyridin-3-yl)-ethanone.

Yield: 98%. LC-MS m/e: 413 (M+H)⁺; t_{R}=1.24. (Method—II).

### EXAMPLE 1f [(Methylethyl)sulfonyl]({trans-4-[(4-(6-fluoro-pyridin-3-yl)(5-methyl 1,3-thiazol-2-yl))amino]cyclohexyl}methyl)amine

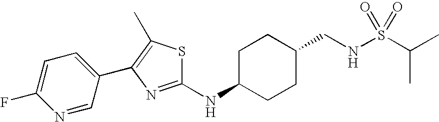

Prepared from N-({[(trans-4-{[(methylethyl)sulfonyl]methylamino}cyclohexyl]amino}thioxomethyl)amide and 2-bromo-1-(6-fluoro-pyridin-3-yl)-propan-1-one HBr salt.

Yield: 77%. LC-MS m/e: 427 (M+H)⁺; t_{R}=1.26 (Method—I).

### EXAMPLE 1g [(Methylethyl)sulfonyl]({trans-4-[(4-(2-fluoro-pyridin-3-yl)(5-methyl-1,3-thiazol-2-yl))amino]cyclohexyl}methyl)amine

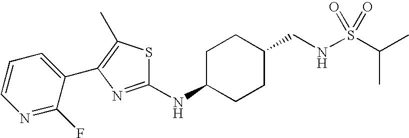

Prepared from N-({[(trans-4-{[(methylethyl)sulfonyl]methylamino}cyclohexyl]amino}thioxomethyl)amide and 2-bromo-1-(2-fluoro-pyridin-3-yl)-propan-1-one HBr salt.

Yield: 55%. LC-MS m/e: 427 (M+H)⁺; t_{R}=1.17 (Method—I).

### EXAMPLE 1h [(Methylethyl)sulfonyl]({4-[(trans-4-(6-trifluoromethyl-pyridin-3-yl)(5-methyl-1,3-thiazol-2-yl))amino]cyclohexyl}methyl)amine

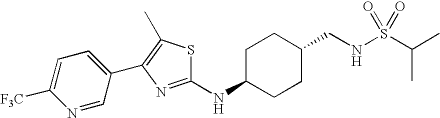

Prepared from N-({[(trans-4-{[(methylethyl)sulfonyl]methylamino}cyclohexyl]amino}thioxomethyl)amide and 2-bromo-1-(6-trifluoromethyl-pyridin-3-yl)-propan-1-one.

Yield: 38%. LC-MS m/e: 477 (M+H)⁺; t_{R}=1.47 (Method—I).

### EXAMPLE 1i [(Methylethyl)sulfonyl](trans-4-{[(4-(6-bromopyridin-2-yl)(1,3-thiazol-2-yl))amino]methyl}cyclohexyl)amine

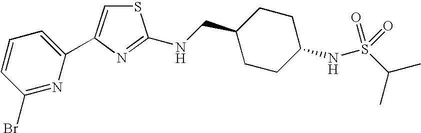

Prepared from N-({[(trans-4-{[(methylethyl)sulfonyl]amino}cyclohexyl)methyl]amino}thioxomethyl)amide and 2-bromo-1-(6-bromo-pyridin-2-yl)-ethanone HBr salt.

Yield: 47%. LC-MS m/e: 475 ((M+H)⁺+2); t_{R}=1.44 (Method—II).

### EXAMPLE 1j [(tert-butyl)sulfonyl]({4-[(trans-4-(6-trifluoromethyl-pyridin-3-yl)(5-methyl-1,3-thiazol-2-yl))amino]cyclohexyl}methyl)amine

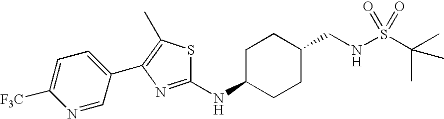

Prepared from N-({[(trans-4-{[(tert-butyl)sulfonyl]methylamino}cyclohexyl]amino}thioxomethyl)amide and 2-bromo-1-(6-trifluoromethyl-pyridin-3-yl)-propan-1-one HBr salt.

Yield: 6.0%. LC-MS m/e: 491 (M+H)⁺; t_{R}=1.56 (Method—II).

The following compound of the invention was prepared from intermediate Formula XV and synthesized according to the procedures described in Scheme 3:

### EXAMPLE 2a [(cyclopropyl)sulfonyl]({4-[(trans-4-(6-trifluoromethyl-pyridin-3-yl)(5-methyl-1,3-thiazol-2-yl))amino]cyclohexyl}methyl)amine

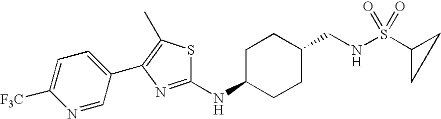

Cyclopropanesulfonyl chloride (0.05 g, 0.48 mM) was added to a solution of the trifluoroacetate salt of [trans-4-(aminomethyl)cyclohexyl](4-(6-trifluoromethyl-pyridin-3-yl)(1,3-thiazol-2-yl))amine (0.10 g) and DIEA (0.2 mL) in CH₂Cl₂ (2 mL) at rt. The reaction was stirred at rt for 10 min. The solvent was removed in vacuo and the product was purified by preparative TLC (50% Hexanes in EtOAc), to afford the title compound (0.015 g, 19%). ¹H NMR (CDCl₃) δ 9.06 (s, 1H), 8.15 (d, 1H, J=14 Hz), 7.79 (d, 1H, J=4.8 Hz), 5.78 (br, m, 1H), 4.70 (t, 1H, J=4.8 Hz), 3.31 (br, m, 1H), 3.10 (t, 2H, J=4.8 Hz), 2.54 (s, 3H), 2.29 (d, 2H, J=14.3 Hz), 1.96 (d, 2H, J=14.0 Hz), 1.60 (br, m, 1H), 1.38-0.94 (m, 8H). LC-MS m/e: 475 (M+H)⁺; t_{R}=1.43 (Method—II).

### Formulations

The pharmaceutical formulations of the invention may be prepared by conventional methods in the art.

For example, tablets may be prepared by mixing the active ingredient with ordinary adjuvants and/or diluents and subsequently compressing the mixture in a conventional tabletting machine may prepare tablets. Examples of adjuvants or diluents comprise: corn starch, potato starch, talcum, magnesium stearate, gelatine, lactose, gums, and the like. Any other adjuvants or additives usually used for such purposes such as colorings, flavorings, preservatives etc. may be used provided that they are compatible with the active ingredients.

1) Tablets containing 5.0 mg of Compound 1b calculated as the free base:

| | | |
|---|---|---|
| Compound | 5.0 | mg |
| Lactose | 60 | mg |
| Maize starch | 30 | mg |
| Hydroxypropylcellulose | 2.4 | mg |
| Microcrystalline cellulose | 19.2 | mg |
| Croscarmellose Sodium Type A | 2.4 | mg |
| Magnesium stearate | 0.84 | mg |

2) Tablets containing 0.5 mg of Compound 1b calculated as the free base:

| | | |
|---|---|---|
| Compound | 0.5 | mg |
| Lactose | 46.9 | mg |
| Maize starch | 23.5 | mg |
| Povidone | 1.8 | mg |
| Microcrystalline cellulose | 14.4 | mg |
| Croscarmellose Sodium Type A | 1.8 | mg |
| Magnesium stearate | 0.63 | mg |

3) Syrup containing 25 mg of Compound 1b per milliliter:

| | | |
|---|---|---|
| Compound | 25 | mg |
| Sorbitol | 500 | mg |
| Hydroxypropylcellulose | 15 | mg |
| Glycerol | 50 | mg |
| Methyl-paraben | 1 | mg |
| Propyl-paraben | 0.1 | mg |
| Ethanol | 0.005 | mL |
| Flavor | 0.05 | mg |
| Saccharin | 0.5 | mg |
| Water | 1 | mL |

### In-Vitro Methods

The pharmacological properties of the compounds of the present invention were evaluated at the cloned human NPY Y5 receptor using the protocols disclosed in U.S. Pat. No. 6,124,331, the contents of which are hereby incorporated by reference.

Using this protocol, the binding by the compound to a radiolabeled ligand (125I-labeled PYY or an alternative radioligand such as ¹²⁵I-labeled NPY) to membranes of cloned human NPY Y5 receptors expressed in COS-7 cells was determined in vitro.

### Radioligand Binding

Membrane suspensions were diluted in binding buffer supplemented with 0.1% bovine serum albumin to yield an optimal membrane protein concentration so that ¹²⁵I-PYY bound by membranes in the assay was less than 10% of ¹²⁵I-PYY delivered to the sample (100,000 dpm/sample=0.08 nM for competition binding assays). ¹²⁵I-PYY and small molecule ligand competitors were also diluted to desired concentrations in supplemented binding buffer. Individual samples were then prepared in 96-well polypropylene microtiter plates by mixing ¹²⁵I-PYY, competing peptides or supplemented binding buffer (25 μL), and finally, membrane suspensions (200 μL). Samples were incubated in at 30° C. for 120 min. Incubations were terminated by filtration over Whatman GF/C filters (pre-coated with 1% polyethyleneimine and air-dried before use), followed by washing with 5 mL of ice-cold binding buffer. Filter-trapped membranes were impregnated with MeltiLex solid scintillant (Wallac, Turku, Finland) and counted for ¹²⁵I-PYY in a Wallac Beta-Plate Reader. Alternatively, incubations were carried out in GF/C filter plates (pre-coated with 1% polyethyleneimine and air-dried before use), followed by vacuum filtration and three washes of 300 μL of ice-cold binding buffer. 50 μL of UltimaGold (Packard) scintillant were added and counted for ¹²⁵I-PYY in a Wallac MicroBeta Trilux. Non-specific binding was defined by 300 nM human PYY. Specific binding in time course and competition studies was typically 80%; most non-specific binding was associated with the filter. Binding data were analyzed using nonlinear regression and statistical techniques available in the GraphPAD Prism package (San Diego, Calif.).

The binding affinities for the compounds in the present invention, exemplified above, at the NPY Y5 receptor were determined to be 50 nM or less. The binding affinities for some of the compounds were determined to be 10 nM or less.

What is claimed:

1. A compound having the structure: 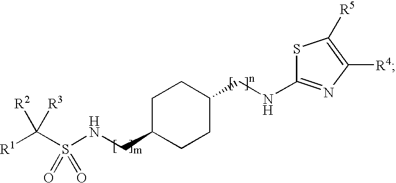
wherein R¹ is H or C₁-C₆ straight chained or branched alkyl;
wherein R² is C₁-C₆ straight chained or branched alkyl;
or wherein R¹, R² and the carbon to which they are attached may form C₃-C₆ cycloalkyl;
wherein R³ is H or methyl;
wherein R⁴ is 2-pyridyl or 3-pyridyl, wherein the 2-pyridyl or 3-pyridyl is substituted with one or more F, Cl, Br, I or CF₃, and optionally substituted with CH₃;
wherein R⁵ is H or methyl;
wherein m is an integer from 0 to 2 inclusive; and
wherein n is an integer from 0 to 2 inclusive;
or a pharmaceutically acceptable salt thereof.

2. The compound of claim 1, wherein R³ is H; wherein m is 0 or 1; and wherein n is 0 or 1.

3. The compound of claim 2, wherein R¹ is H or C₁-C₄ straight chained or branched alkyl.

4. The compound of claim 3, wherein R² is C₁-C₄ straight chained or branched alkyl.

5. The compound of claim 4, wherein R¹ is H, methyl or ethyl; and wherein R² is methyl or ethyl.

6. The compound of claim 5, wherein R¹ is methyl; R² is methyl; and R⁴ is 2-pyridyl or 3-pyridyl, wherein the 2-pyridyl or 3-pyridyl is substituted with F or CF₃ and optionally substituted with CH₃.

7. The compound of claim 6, wherein n is 1.

8. The compound of claim 6, wherein m is 1.

9. The compound of claim 7, wherein R⁴ is 3-pyridyl substituted with F; and wherein R⁵ is H.

10. The compound of claim 8, wherein R⁴ is 3-pyridyl substituted with F; and wherein R⁵ is H.

11. The compound of claim 2, wherein R¹, R² and the carbon to which they are attached form C₃-C₆ cycloalkyl.

12. The compound of claim 11, wherein R⁴ is 2-pyridyl or 3-pyridyl substituted with F.

13. The compound of claim 12, wherein R¹, R² and the carbon to which they are attached form cyclopropyl or cyclobutyl; wherein R⁴ is 3-pyridyl substituted with F; wherein R⁵ is H; and wherein n is 0.

14. The compound of claim 1, wherein the compound is selected from the group consisting of [(methylethyl)sulfonyl](trans-4-{[(4-(6-fluoro-pyridin-2-yl)(1,3-thiazol-2-yl))amino]methyl}cyclohexyl)amine; [(methylethyl)sulfonyl]({trans-4-[(4-(6-fluoro-pyridin-2-yl)(1,3-thiazol-2-yl))amino]cyclohexyl}methyl)amine; [(methylethyl)sulfonyl]({trans-4-[(4-(2-methyl,6-fluoro-pyridin-3-yl)(1,3-thiazol-2-yl))amino]cyclohexyl}methyl)amine; and (methylethyl)sulfonyl](trans-4-{[(4-(2-fluoro-pyridin-3-yl)(1,3-thiazol-2-yl))amino]methyl}cyclohexyl)amine or a pharmaceutically acceptable salt thereof.

15. The compound of claim 1, wherein the compound is selected from the group consisting of [(methylethyl)sulfonyl]({trans-4-[(4-(2-fluoro-pyridin-3-yl)(1,3-thiazol-2-yl))amino]cyclohexyl}methyl)amine; [(methylethyl)sulfonyl]({trans-4-[(4-(6-fluoro-pyridin-3-yl)(5-methyl-1,3-thiazol-2-yl))amino]cyclohexyl}methyl)amine; and (methylethyl)sulfonyl]({trans-4-[(4-(2-fluoro-pyridin-3-yl)(5-methyl-1,3-thiazol-2-yl))amino]cyclohexyl}methyl)amine or a pharmaceutically acceptable salt thereof.

16. The compound of claim 1, wherein the compound is selected from the group consisting of [(methylethyl)sulfonyl]({trans-4-[(4-(6-trifluoromethyl-pyridin-3-yl)(5-methyl-1,3-thiazol-2-yl))amino]cyclohexyl}methyl)amine; and [(methylethyl)sulfonyl](trans-4-{[(4-(6-bromopyridin-2-yl)(1,3-thiazol-2-yl))amino]methyl}cyclohexyl)amine or a pharmaceutically acceptable salt thereof.

17. A pharmaceutical composition comprising a therapeutically effective amount of a compound of claim 1 and a pharmaceutically acceptable carrier.

18. A method of treating a subject suffering from depression comprising administering to the subject a therapeutically effective amount of a compound of claim 1.

19. A method of treating a subject suffering from anxiety comprising administering to the subject a therapeutically effective amount of a compound of claim 1.

20. A method of treating a subject suffering from obesity comprising administering to the subject a therapeutically effective amount of a compound of claim 1.
